(19) United States
(12) Patent Application Publication
(10) Pub. No.: US 2016/0192852 A1
(43) Pub. Date: Jul. 7, 2016

(54) WIRELESS CARDIAC EVENT RECORDER

(71) Applicant: EVENT CARDIO GROUP, INC., Boyton Beach, FL (US)

(72) Inventors: Nicholas D. Bozza, Thorold (CA); Gianfranco Bentivoglio, Thorold (CA); Ricardo Alberto Rodriguez Cornejo, Richmond Hills (CA)

(21) Appl. No.: 14/392,259

(22) PCT Filed: Jun. 25, 2014
(86) PCT No.: PCT/CA2014/000518

§ 371 Date: Dec. 24, 2015

Related U.S. Application Data
(60) Provisional application No. 61/838,779, filed on Jun. 24, 2013.

(51) Int. Cl. A61B 5/0432; A61B 5/0408; A61B 5/0404; A61B 5/00

(52) U.S. Cl. CPC A61B 5/0432; A61B 5/0006; A61B 5/04085; A61B 5/0404; A61B 5/6832

(57) ABSTRACT

A wireless cardiac event recorder is provided comprising two or more electrodes capable of detecting a patient's heart beat and producing a corresponding electronic signal. A transmitter component is coupled to the electrodes to receive the electronic signal and wirelessly transmit the signal to a cardiac event monitor.

### FIELD OF THE INVENTION

This invention generally relates to embodiments of a new wireless cardiac event recorder.

### DESCRIPTION OF THE PRIOR ART

Cardiac monitoring may be important when a physician suspects that a patient has a cardiac problem, but cannot detect any irregular cardiac symptoms in the office or hospital. There are generally two methods for monitoring cardiac outputs. The first type of cardiac monitor or ambulatory electrocardiography device is known as a Holter recorder, used for the continuous recording of a patient's cardiac output. The second type of cardiac monitor is a loop event recorder (LER) which monitors the patient 24 hours per day however only records based on pre-set programming for anomalies and/or a patient trigger.

When used for the heart, (much like standard electrocardiography) a Holter monitor records electrical signals from the heart via a series of electrodes attached to the chest. Electrodes are placed are placed in certain places on the skin over bones to minimize artifacts from muscular activity. The number and position of electrodes varies by model, but most Holter monitors employ between three and eight while LER's employ 2 to 3 electrodes. These electrodes are connected to a small piece of equipment that is attached to the patient's belt or hung around the neck, and is responsible for keeping a log of the heart's electrical activity throughout the recording period.

The loop event recorder does not continuously store data; rather, it only stores a short record (30 seconds to five minutes-choice based on patient symptoms) when a certain condition occurs; whether it be a prompt from a patient, or the occurrence of a designated threshold value. For example, when a patient senses an event or abnormal condition coming on, the patient may press an event button so that a cardiac reading can be captured and stored while the patient experiences this condition or event.

A symptom event recorder (non-looping) can be either a hand held device or worn on a patient's wrist. When the patient feels a symptom or irregular heartbeat, the patient places the monitor on their chest and activates a recording button. The back of the device has small metal discs that function as the electrodes.

Monitors that use electrodes connected to a monitor or other recording device are uncomfortable and may not be worn while bathing, showering or swimming. Patient adherence is usually very poor unless the facilitators have an extensive patient usage program. It is contended that patients who do not attend a proper patient fitting and usage program (minimum 30 minutes) do not wear the device for the full period of time that it is required. Hence much important data may be missed and based on this, incorrect treatment may be followed because of the un-optimal diagnosis. In short more patients may be hurt rather than helped.

There is a need for a wireless receiver that has real time correlations with other biometric measurements such as blood pressure and can play back earlier cardiac event data.

Further there is a need for electrodes that are waterproof and can be worn when taking a shower or other moisture conditions such as perspiration.

### SUMMARY OF THE INVENTION

The present disclosure provides embodiments of a wireless cardiac event recorder that may be used with existing monitors and cardiac event recorders but without any wires from the electrodes to the monitor or recording device.

In embodiments disclosed a wireless cardiac event recorder is provided comprising two or more electrodes capable of detecting a patient's heart beat and producing a corresponding electronic signal. A transmitter component is coupled to the electrodes to receive the electronic signal and wirelessly transmit the signal to a cardiac event monitor

Further features of the embodiments of a wireless cardiac event recorder will be described or will become apparent in the course of the following detailed description.

### BRIEF DESCRIPTION OF THE DRAWINGS

In order that the invention may be more clearly understood, embodiments thereof will now be described in detail by way of example, with reference to the accompanying drawings, in which:

FIG. 1 is a perspective view of one embodiment of a wireless cardiac event recorder according to the disclosure having electrodes and a transmitter component, a separate receiver component and a portable monitor or event recorder.

FIG. 2 is a block diagram of the electrodes and transmitter component of FIG. 1;

FIG. 3 is a schematic plan view of the top of the circuit board for the transmitter component of FIG. 2;

FIG. 4 is a schematic plan view of the bottom of the circuit board for the transmitter component of FIG. 2;

FIG. 5 is a block diagram of the receiver component of FIG. 1;

FIG. 6 is a schematic plan view of the top of the circuit board for the receiver component of FIG. 4;

FIG. 7 is a schematic plan view of the bottom of the circuit board for the receiver component of FIG. 4;

FIG. 8 is a flow diagram for the functionality of the transmitter component and the receiver component of FIG. 1.

FIG. 9 is a schematic plan view of the top of another embodiment of a circuit board for the transmitter component according to the disclosure herein;

FIG. 10 is a schematic plan view of the top of another embodiment of a circuit board for the receiver component according to the disclosure herein;

FIG. 11 is a perspective view of another embodiment of a wireless cardiac event recorder according to the disclosure adapted to affix to an individual having electrodes and a housing containing processing means, a transmitter, a receiver and a power source.

Similar references are used in different figures to denote similar components.

### DETAILED DESCRIPTION

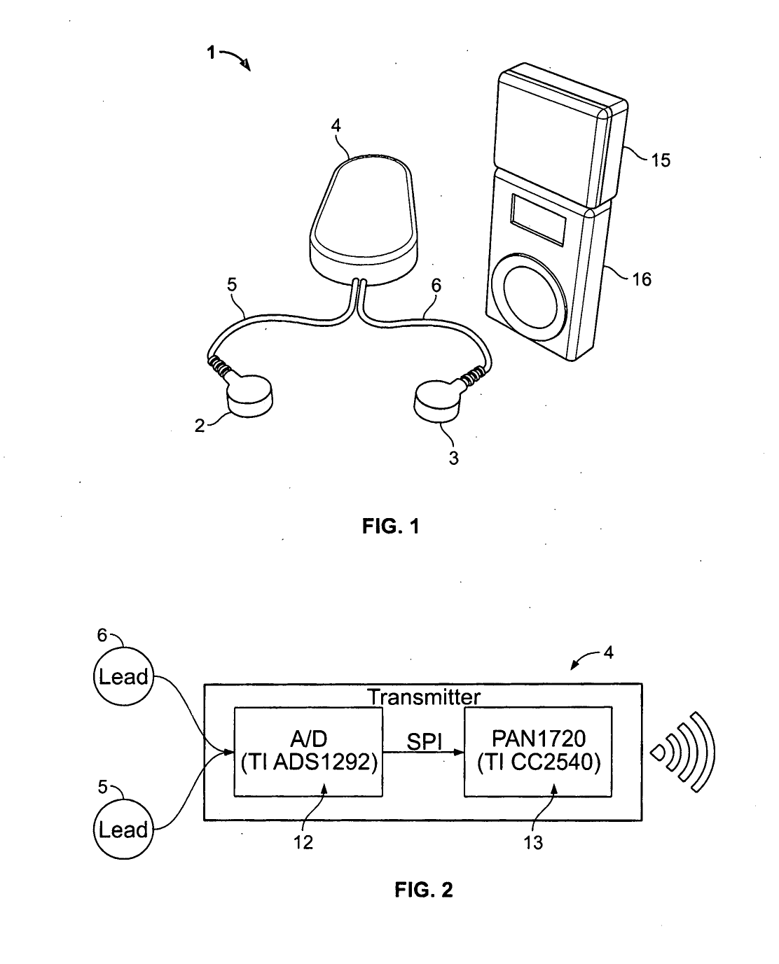

Referring to FIG. 1, one embodiment of a wireless cardiac event recorder according to the present disclosure is generally indicated at 1. The wireless cardiac event recorder 1 has a pair of electrocardiogram (ECG) capable electrodes 2,3 connected to a transmitter component 4. In the embodiment shown, the electrodes 2,3 are shown separate from the transmitter component 4 and connected by leads 5,6 to the transmitter component 4. Electrodes 2,3 and transmitter component 4 are adapted to be placed against the patient's body and retained in position. Suitable electrodes can be obtained from a number of suppliers including 3M™'s Red Dot™ diagnostic ECG electrodes. These electrodes have a pressure sensitive adhesive that enables the electrodes to be applied to and adhered to a patient's skin. Electrodes 2,3 monitor the patient's heart beat and send an ECG signal to the transmitter component through leads 5,6. The electrodes 2,3 are placed on the RA and V₅ positions. The “RA” position is located on the right arm at the wrist or shoulder and the “V₅” position is located In the fifth intercostal space (between ribs 5 and 6) in the left anterior axillary line.

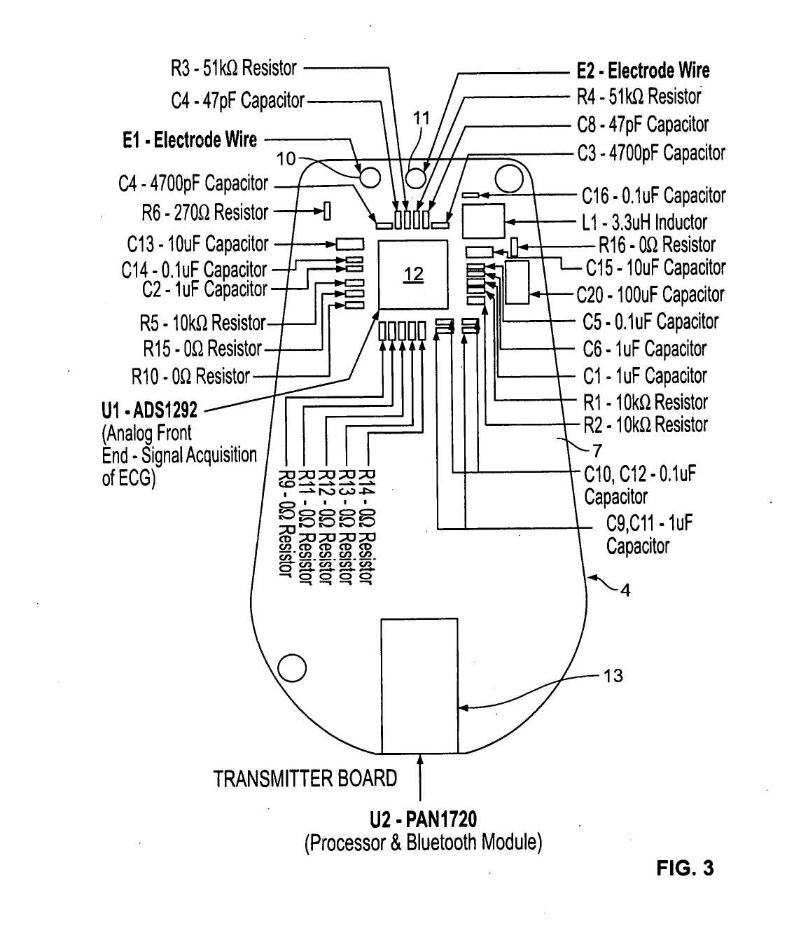

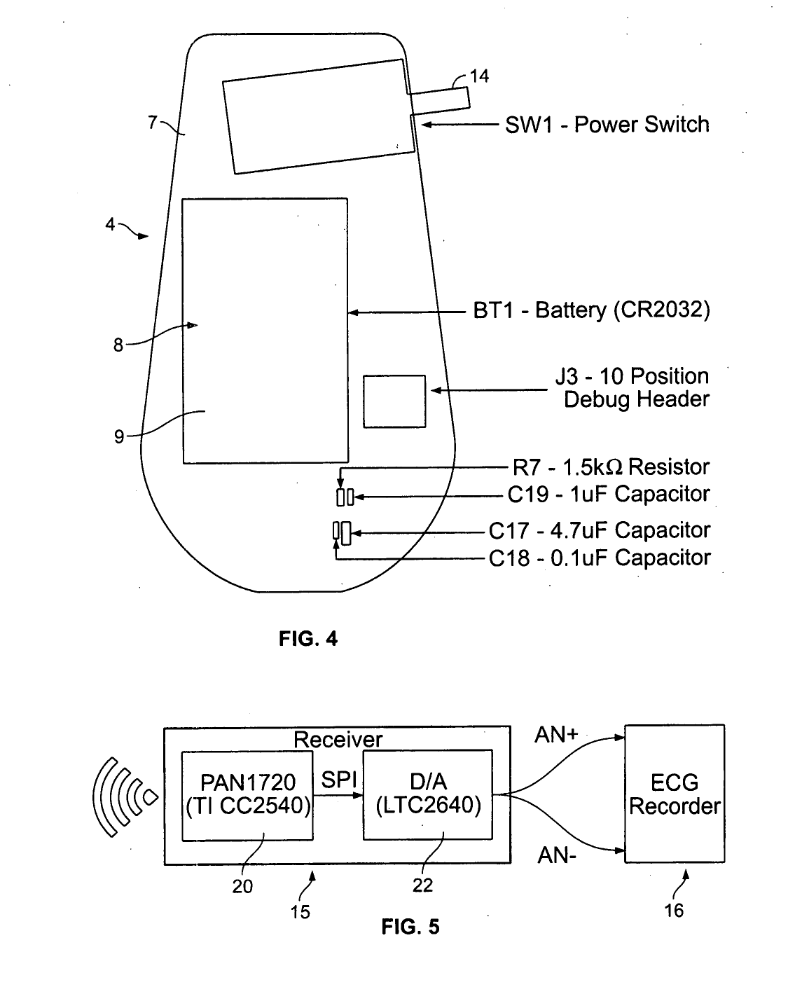

As shown in FIGS. 2-4 transmitter component 4 has a circuit board 7 with a power means generally indicated at 8. In the embodiment illustrated power means 8 is a battery 9. The leads 5,6 are connected to the circuit board 7 at terminals 10, 11. Referring to FIGS. 3 and 4, as the ECG signal from the electrodes 2,3 may be an analog signal, an analog/digital converter 12 is provided to capture the ECG signal and convert it to a digital signal and then transmitting it over a Serial Peripheral Interface (SPI) bus to the microprocessor 13 with wireless transceiver and optional data storage. The wireless transceiver wirelessly transmits the digital data from the microprocessor 13 to a receiver component 15 (shown in FIG. 1). An on/off switch 14 is provided.

In the embodiment illustrated electrodes 2,3 are shown separate from transmitter component 4, however the electrodes 2,3 may be connected to the back side of a similar circuit board with the electrical components on the front side of the circuit board, in the form of a self-adhering patch. In this embodiment the electrodes 2,3 should be spaced apart to obtain the appropriate ECG signal.

Alternatively the individual electrodes 2,3 can each be made with a power source (cell battery), LED lights to indicate when the electrode is powered on or off, on/off switches and a built in electronics including analog/digital converter, microprocessor, data storage and transmitter. This embodiment eliminates the need for transmitter component 4.

Electrodes 2,3 may be waterproof, easily cleaned and possibly sterilized between uses to permit the patient to retain the electrodes on the skin when showering and for reuse. The transmitter component 4 can be waterproof to permit the patient to shower.

In the embodiment illustrated the receiver component 15 is adapted to mount to a known cardiac event recorder 16 such as the King of Hearts™ cardiac event recorder or other monitor or cardiac event recorder. It receives the ECG signal from the transmitter component 4, converts the digital signal back into an analog signal and the signal is provided to the recording device 16.

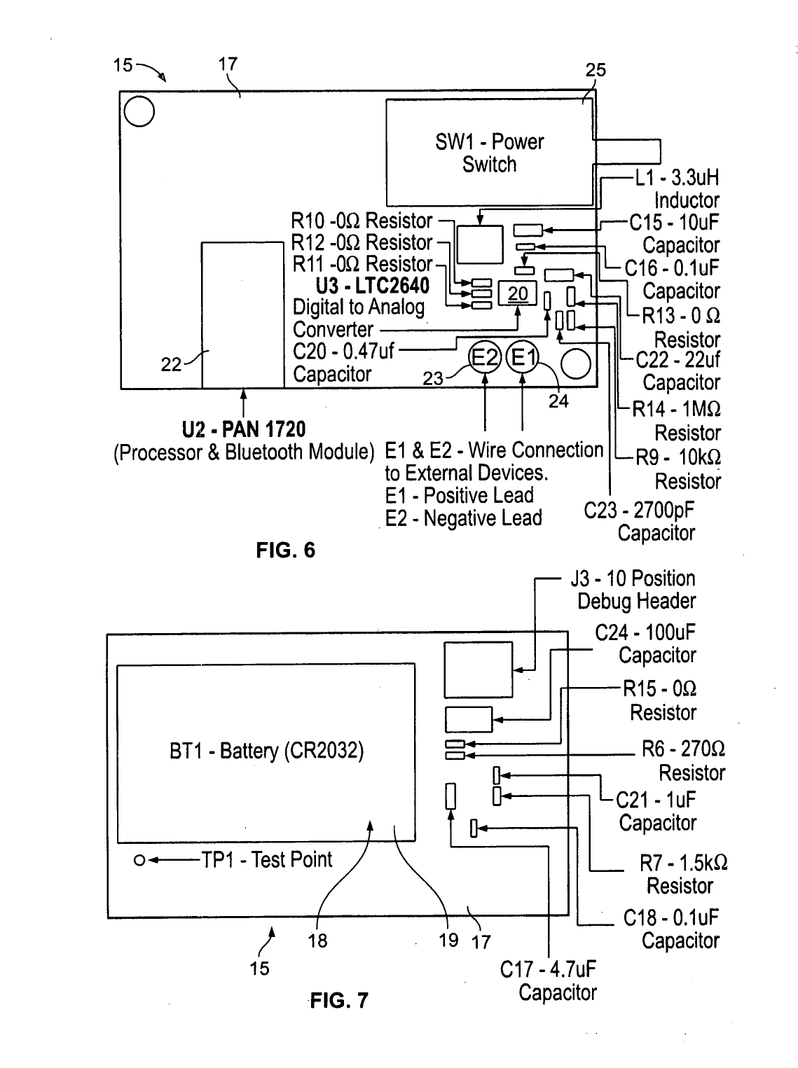

As shown in FIG. 5-7 receiver component 15 has a circuit board 17 with a power means generally indicated at 18 shown as battery 19. Referring to FIG. 6, as the digital signal from the transmitter component 4 maybe a digital signal, a digital/analog converter 20 is provided to convert back to a analog signal and then transmitting it over a Serial Peripheral Interface (SPI) bus to a microprocessor 21. The receiver component 15 interfaces with the external monitor or event recorder to receive the ECG data from the microprocessor 22. In the embodiment illustrated external devices are connected to receiver component 15 at terminals 23,24. A power switch 25 is provided for on receiver component 15.

The embodiment illustrated can either continuously record data once activated or be programmed to act as a cardiac loop recorder or event recorder.

While receiver component 15 is shown as a separate unit from the cardiac event recorder or monitor it can be built into the cardiac event recorder or monitor.

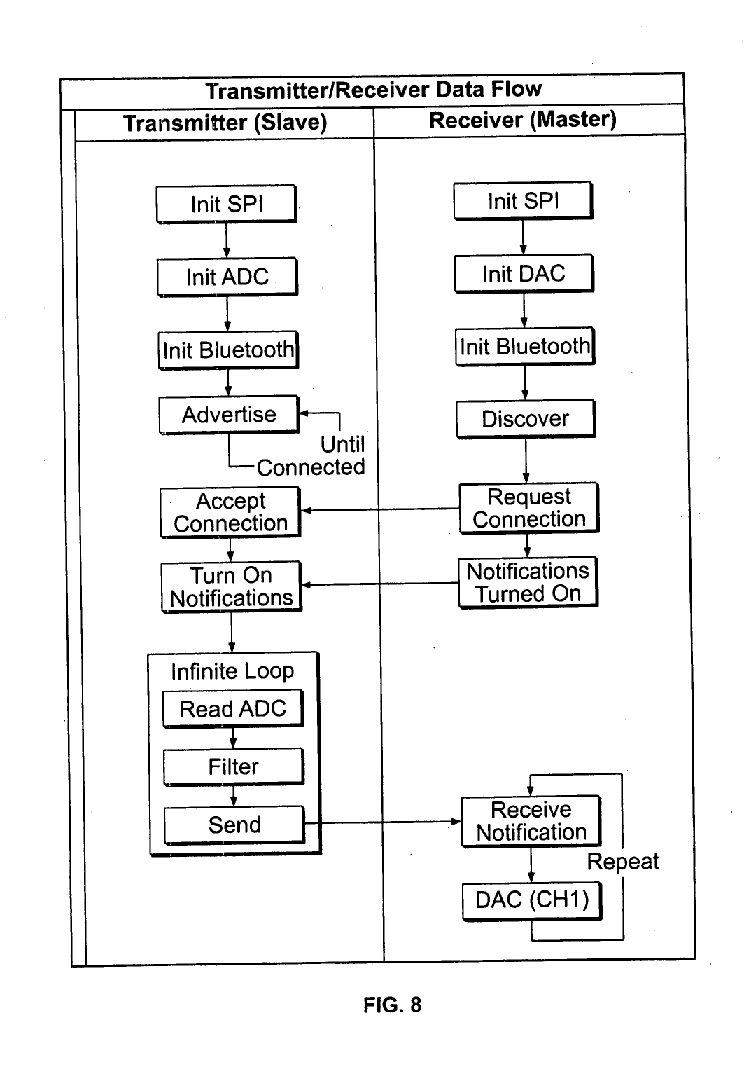

FIG. 8 shows a flow diagram of how the wireless cardiac event recorder 1 operates. The receiver component 15 is connected to the event cardiac recorder 16 causing the event cardiac recorder 16 to be turned on. In FIG. 8 the transmitter component 4 and receiver component 15 are first powered on by depressing a power switch on both the transmitter component 4 and receiver component 15. Green LED lights on the transmitter component 4 will flash three times to indicate the transmitter component is turned on. Depressing the power switch of receiver component 15 automatically commences initialization of the SPI, analog/digital converter and wireless transceiver on the transmitter component as well as commencing initialization of the SPI, digital/analog converter and wireless transceiver on the receiver component. The receiver component sends a request for connection to the transmitter component. On acceptance of the connection request (may be automatic or manual), notifications are turned on. A green LED light will flash three times once the connection with the transmitter component has been established. The transmitter component and the electrodes are now ready to read and transmit the ECG signal. The transmitter component sends the ECG signal to the receiver component over the wireless connection. In FIG. 6 the wireless connection is shown as a Bluetooth connection.

In the embodiment illustrated in the Figures, both the transmitter component 4 and receiver component 15 are factory programmed. However, modifications are possible to convert both units so that they can be programmed at the time of first use to personalize the device for the particular patient to be monitored.

The factory programming of the embodiment illustrated is intended to be compatible to the cardiac event recorder 16 which will record and store data either continuously or in a loop. The cardiac event recorder 16 is programmed to automatically record when a certain anomaly in patient heart beat such as arrhythmias are detected or if patient initiates event recording.

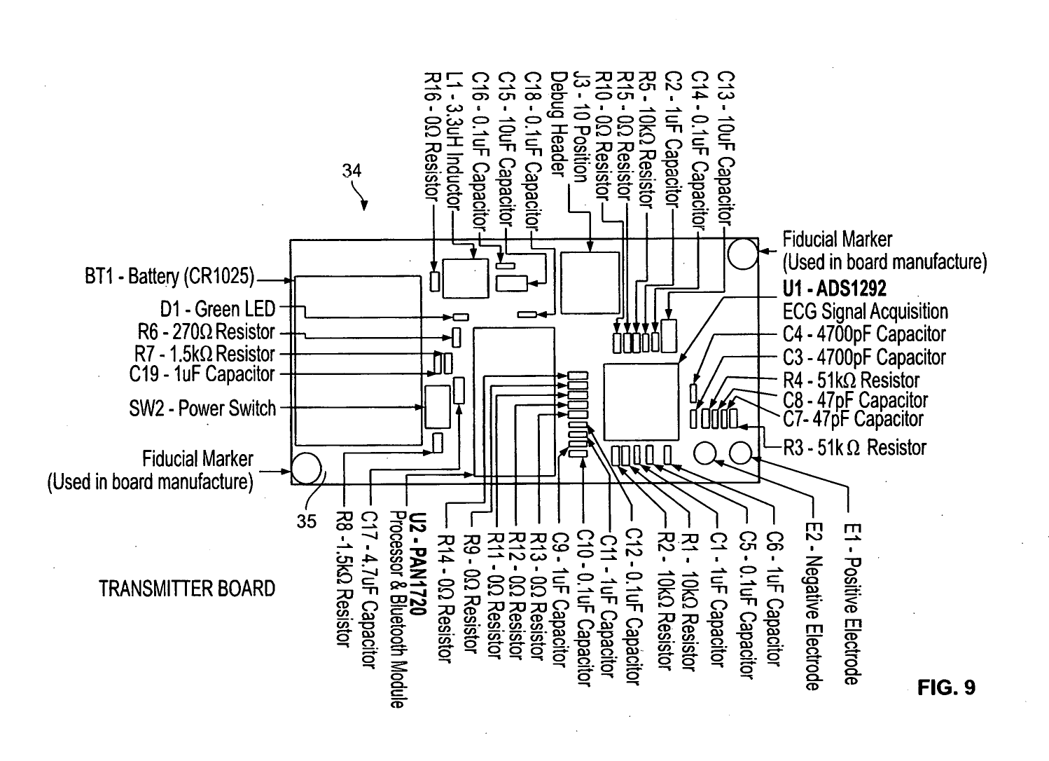

FIG. 9 illustrates another embodiment of a transmitter component 34 having similar electronic components as for the embodiment shown in FIGS. 2- 4 but located on one side of the circuit board 35.

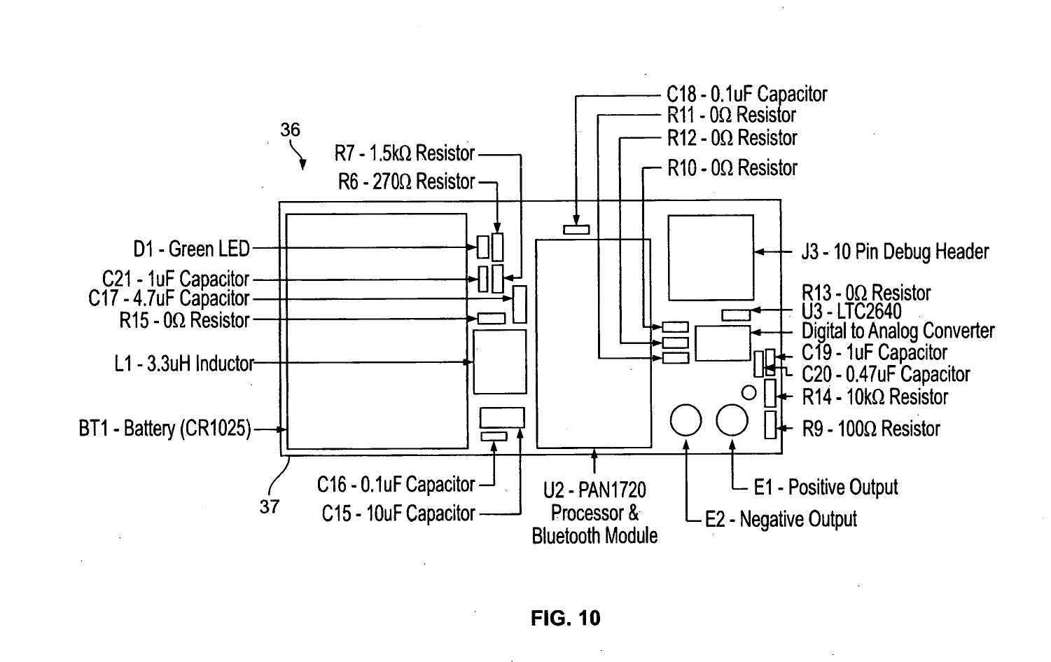

FIG. 10 illustrates another embodiment of a receiver component 36 having similar electronic components as for the embodiment shown in FIGS. 5- 7 but located on one side of the circuit board 37.

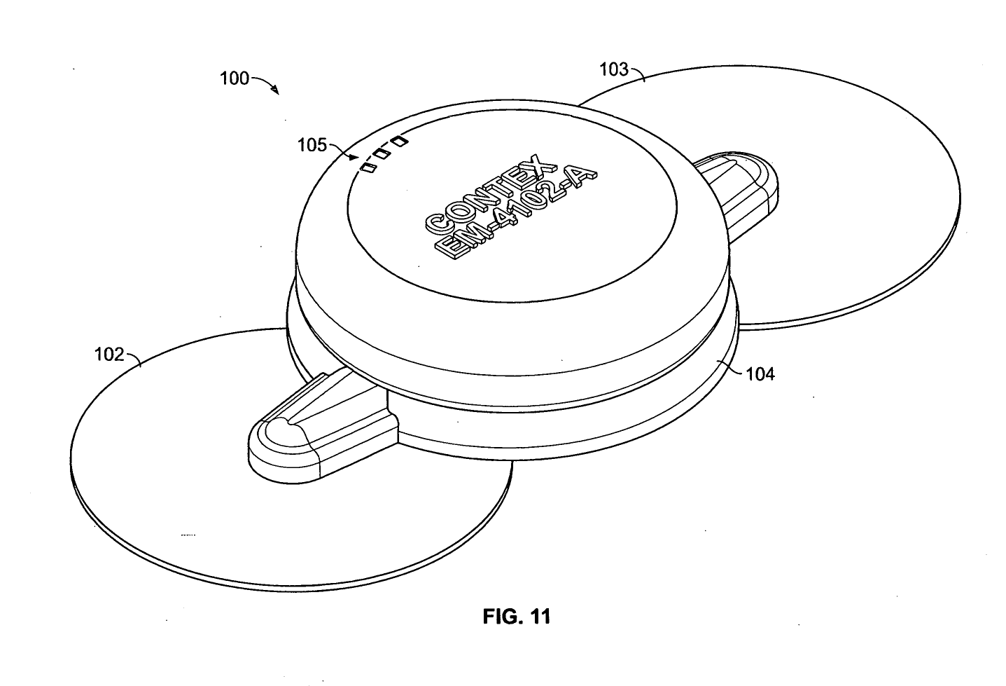

Referring to FIG. 11, another embodiment of a wireless cardiac event recorder according to the present disclosure is generally indicated at 100. The wireless cardiac event recorder 100 has a pair of electrocardiogram (ECG) capable electrodes 102, 103 connected to a housing 104. In the embodiment shown, the electrodes 102,103 are shown connected to the back side of housing 104 and connected by leads (not shown) to a microprocessor with transmitter/receiver on a circuit board within the housing. Electrodes 102,103 and housing 104 are adapted to be placed against the patient's body and retained in position. Suitable electrodes can be obtained from a number of suppliers including 3M™'s Red Dot™ diagnostic ECG electrodes. These electrodes have a pressure sensitive adhesive that enables the electrodes to be applied to and adhered to a patient's skin.

Electrodes 102,103 monitor the patient's heart beat and send an ECG signal to the processor/transmitter in the housing 104.

As noted above within housing 104 is a circuit board with a power means. In the embodiment illustrated power means is a battery. The leads are connected to the circuit board. The ECG signal from the electrodes 102,103 may be an analog signal, so an analog/digital converter is provided on the circuit board to capture the ECG signal and convert it to a digital signal. The ECG signal is sent to the microprocessor with wireless transceiver and optional data storage. The wireless transceiver wirelessly transmits the digital data from the microprocessor to a Smartphone for transmission to a data centre. An on/off switch is provided.

LED lights 105 indicate when the unit is powered on or off, on/off switches and a built in electronics including analog/digital converter, microprocessor, data storage and transmitter.

The electrodes 102, 103 and housing 104 may be waterproof, easily cleaned and possibly sterilized between uses to permit the patient to retain the electrodes on the skin when showering and for reuse.

The embodiment illustrated can either continuously record data once activated or be programmed to act as a cardiac loop recorder or event recorder.

Potential features of the embodiment illustrated in FIG. 11 include: (1) providing data storage on the circuit board that will use for a period of time without loss of data and when back in range to Smartphone transmit the stored data (2) built in inputs for other biometric data (3) low battery detection (4) audio outputs (5) Providing additional firmware to auto-detect patient episodes of atrial fibrillation, Bradycardia, Tachycardia, and pause, with user notifications on occurrence of a cardiac event (6) providing modification that will enable data streaming to a smart phone or smart device (7) providing modification to use 3.6V Lithium Ion rechargeable battery.

While the principles of the invention have been shown and described in connection with specific embodiments, it is to be understood that such embodiments are by way of example and are not limiting As is evident from the foregoing description, certain aspects of the present invention are not limited by the particular details of the mobile stand illustrated in the drawings. Other modifications and applications, or equivalents, will occur to those skilled in the art. The terms “having”, “comprising” and “including” and similar terms as used in the foregoing specification are used in the sense of “optional” or “may include” and not as “required”. Many changes, modifications, variations and other uses and applications of the present construction will, however, become apparent to those skilled in the art after considering the specification and attached drawings. Potential modifications include (1) providing data storage on the transmitter component 4 or electrodes 2,3 that will enable the patient to be separated from the receiver component 15 for a period of time without loss of data and when back in range transmit the stored data (2) built in inputs for other biometric data (3) low battery detection (4) audio outputs and (5) user notifications on occurrence of a cardiac event. All such changes, modifications, variations and other uses and applications which do not depart from the spirit and scope of the invention are deemed to be covered by the invention which is limited only by the claims that follow. The scope of the disclosure is not intended to be limited to the embodiments shown herein, but is to be accorded the full scope consistent with the claims, wherein reference to an element in the singular is not intended to mean “one and only one” unless specifically so stated , but rather one or more. All structural and functional equivalents to the elements of the embodiment described throughout the disclosure that are known or later come to be known to those of ordinary skill in the art are expressly incorporated herein by reference and intended to be encompassed by the claims.

The embodiments of the invention in which an exclusive property or privilege is claimed are defined as follows:

1. A wireless cardiac event recorder comprising two or more electrodes capable of detecting a patient's heart beat and produce a corresponding electronic signal, a transmitter component coupled to said electrodes to receive the electronic signal, said transmitter component wirelessly transmitting the signal to a cardiac event monitor.

2. A wireless cardiac event recorder according to claim 1 wherein the transmitter component comprises a circuit board with a power means, an analog/digital converter to capture the electronic signal from the electrodes and convert it to a digital signal, a microprocessor with optional data storage and a wireless transceiver for wireless transmitting the digital data from the microprocessor to a cardiac event monitor.

3. A wireless cardiac event recorder according to claim 2 further comprising a separate receiver component adapted to mount to a cardiac event recorder and to wirelessly receive the digital data from the transmitter component.

4. A wireless cardiac event recorder according to claim 3 wherein the receiver component has a circuit board with a power means, a transceiver, a digital/analog converter to convert the digital data back to an analog signal and a microprocessor.

5. A wireless cardiac event recorder according to claim 4 wherein the receiver component connects to a cardiac event monitor.

6. A wireless cardiac event recorder according to claim 1 wherein the two or more electrodes and transmitter component are waterproof.

7. A wireless cardiac event recorder according to claim 1 wherein the two or more electrodes and transmitter component are in the form of a self-adhesive patch.

8. A wireless cardiac event recorder according to claim 2 wherein the cardiac event monitor is a Holter monitor.
